(19) United States
(12) Patent Application Publication
(10) Pub. No.: US 2025/0331922 A1
(43) Pub. Date: Oct. 30, 2025

(54) MEDICAL PROCEDURES AND SYSTEMS USING ACTIVE IMPLANTS

(71) Applicant: INTUITIVE SURGICAL OPERATIONS, INC., Sunnyvale, CA (US)

(72) Inventor: Tico Blumenthal, Los Altos, CA (US)

(21) Appl. No.: 18/866,491

(22) PCT Filed: May 17, 2023
(86) PCT No.: PCT/US2023/022491

§ 371 Date: Nov. 15, 2024

Related U.S. Application Data
(60) Provisional application No. 63/343,257, filed on May 18, 2022.

(51) Int. Cl. A61B 34/10; A61B 90/00; A61F 2/95

(52) U.S. Cl. CPC A61B 34/10; A61B 90/06; A61B 90/37; A61F 2/95; A61B 2034/105; A61B 2034/107; A61F 2250/0096

(57) ABSTRACT

Medical procedures, systems and related methods are disclosed. In some examples, a system may include an instrument for delivery of an active implant and at least one processor configured to receive information describing a pose of the active implant, determine a location of the active implant within an anatomical model based on the information describing a location of the active implant, and establish the location of the active implant as a reference within the anatomical model. In some examples, a method may include obtaining an anatomical model of a subject anatomy, determining a pose of the active implant, and registering the anatomical model to a reference frame of an instrument based on the pose of the active implant.

### CROSS-REFERENCED APPLICATIONS

This application claims priority to and benefit of U.S. Provisional Application No. 63/343,257, filed May 18, 2022 and entitled “Medical Procedures and Systems Using Active Implants,” which is incorporated by reference herein in its entirety.

### FIELD

Disclosed examples are related to medical procedures and systems using active implants.

### BACKGROUND

Treatment of certain medical conditions may involve multiple sequential treatments and/or procedures. For example, an initial procedure might be done to perform one or more diagnostic procedures on an anatomical target to determine if medical intervention might be needed. This may include procedures such as the removal of a segment of the lung of a subject which may involve multiple sequential procedures involved with the identification and subsequent treatment of the subject's lung. Other types of procedures that may involve multiple sequential procedures include radiation therapy and repeat therapeutic injections applied to a specific anatomical target multiple times at different time points. Of course, while a few specific procedures are described above, there are any number of medical procedures that may involve interacting with a desired anatomical target during multiple sequential procedures.

### SUMMARY

In some embodiments, a system for performing a medical procedure may include: an instrument for delivery of an active implant within a subject anatomy; and at least one processor configured to: receive an anatomical model of the subject anatomy, wherein the anatomical model is based on data captured prior to delivery of the active implant; receive positional information for the active implant, wherein the positional information includes a pose of the active implant within the subject anatomy; and establish the pose of the active implant as a reference within the anatomical model.

In some embodiments, a method for providing guidance using an anatomical model of a subject anatomy may include: receiving the anatomical model; determining a pose of an active implant delivered to the subject anatomy, wherein the anatomical model is based on data captured prior to delivery of the active implant to the subject anatomy; updating the anatomical model to establish the active implant as a reference within the model based on the pose of the active implant.

In some embodiments, a non-transitory computer-readable storage medium may include processor executable instructions encoded thereon that when executed perform the above method.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting examples when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 illustrates a medical instrument system, in accordance with embodiments of the present disclosure.

FIG. 2 is a flowchart illustrating an exemplary process of using exemplary systems in an initial procedure, in accordance with embodiments of the present disclosure.

FIG. 3A shows a medical instrument system being driven towards a target segment of an anatomy of a subject;

FIG. 3B shows the medical instrument system of FIG. 3A interacting with the target segment of the anatomy of the subject;

FIG. 3C shows the medical instrument system of FIG. 3A delivering an active implant in a desired location relative to the target segment of the anatomy of the subject;

FIG. 4 is a flowchart illustrating an exemplary method performed by exemplary systems, in accordance with embodiments of the present disclosure.

FIG. 5 illustrates an exemplary bronchial anatomy showing anatomical structure, a target segment of the anatomy, and exemplary systems, in accordance with embodiments of the present disclosure.

FIG. 6 is a zoomed in view of FIG. 5, in accordance with embodiments of the present disclosure.

FIG. 7 illustrates an exemplary bronchial anatomy showing anatomical structures, a target segment of the anatomy, and visual guidance such as suture and/or cut lines as well as a treatment instrument pose to aid a user, in accordance with embodiments of the present disclosure.

FIG. 8 is another flowchart illustrating an exemplary method performed by exemplary systems, in accordance with embodiments of the present disclosure.

FIG. 9 is a schematic of an exemplary teleoperated medical system, in accordance with embodiments of the present disclosure.

### DETAILED DESCRIPTION

In certain applications, a target segment of tissue needs to be located, and this can be difficult due to variations in anatomy and the dynamic nature of some organs, such as lungs and the gastro-intestinal tract. Moreover, pre-operative imaging becomes inaccurate as organs move and/or a segment moves within an overall organ, such as may occur with a lung tumor moving as the corresponding portions of the lungs move.

The above problem may become more pronounced over long periods of time as may often occur between an initial medical procedure in which imaging occurs (such as a biopsy visit) and the follow-up medical procedure in which surgery or other operations occur. For example, a suspected cancerous lesion is often biopsied in an initial medical procedure to confirm a positive or negative diagnosis (malignant or benign). Once confirmed as positive, a follow-up medical procedure can be performed to treat the cancerous lesion. Various treatments can be used depending on the particular medical diagnosis. In some cases, the identified lesion can be treated with ablation or drug therapy. In other cases, removal of a segment of tissue is the appropriate treatment. For example, in the case of lung cancer, removal of a segment of the lung is known as a segmentectomy or wedge resection. Treatments like a segmentectomy or wedge resection can be challenging, especially when performed on anterior or posterior basal lobes. For example, the target segment can be difficult to locate due to variations in pulmonary airway and vascular anatomy, and/or because the tissue is hard to differentiate from other, non-target tissue (especially when too small for a surgeon to palpate or sec). Critical sensitive associated structures within or proximate the target segment may also require a high degree of caution by the practitioner. Accordingly, knowing the location of the target segment before such an operation makes the operation safer and more efficient. Of course, other types of procedures may also involve repeated localization of a region of interest including, for example, radiation therapy, repeat therapeutic injections, and other repeated procedures for a desired anatomical target etc.

When medical treatments, such as a segmentectomy, are done over a series of separate medical procedures and/or relocalization of a region of interest is desired, various techniques may be used to help identify target tissue during the sequential procedures. However, there are limitations associated with these various techniques. For example, dye is often conventionally used for surgical localization, but it only lasts hours before it dissipates and becomes unusable, making it unsuitable when the initial procedure is days or weeks before a follow-up procedure (e.g., a common period of time is 1-2 weeks).

In view of the above, the inventor has recognized and appreciated the benefits associated with providing guidance in locating target tissue during medical treatment procedures where the target tissue has been identified in a separate initial diagnostic procedure. Accordingly, an active implant that is configured to provide positional information of the active implant to correspond with an anatomical model of a desired portion of a subject can be used to correlate the same anatomical model to patient anatomy at different times during different procedures. In some examples the positional information provides a location, an orientation, and an implant state including information used to measure scale, translation, and rotation of the active implant within the anatomical structure in which the active implant is implanted. Thus, by employing the active implant as described in some embodiments herein, various improvements to localization can be made, with resulting improvements to surgical operations. For example, in addition to providing a model for guidance for follow up procedures, the inventor has recognized and appreciated that a therapeutic localization sensor of a medical instrument system used to perform the desired follow up procedure can be registered to the anatomical model and real time navigational guidance can be provided. Additionally, by identifying deformation of the implant within the anatomy using the implant state information, any changes experienced by the anatomy between an initial procedure in which the implant is deployed and the follow up procedure can be used to update the anatomical model to reflect changes in the anatomy, providing for a more accurate model. Thus, medical localization can be completed faster and/or more accurately employing an active implant that is delivered to a desired location within a subject's anatomy than is conventionally possible.

In some embodiments, a method for providing guidance during a medical procedure using an anatomical model (e.g., a three-dimensional anatomical model) may include obtaining the anatomical model of the subject anatomy. This may include either generating a model as described below and/or obtaining a previously generated model. Positional information (e.g., a location, orientation, implant state) of an active implant disposed within the subject's anatomy may then be determined. As elaborated on further below, this may include the use of various sources of information including, but not limited to, instrument localization sensors during deployment of the active implant, user input, the location of target tissue relative to the active implant, implant localization sensor information, combinations of the foregoing, and/or any other appropriate information source. The anatomical model of the subject may then be updated based on the determined positional information of the active implant. For example, the pose of the active implant may be used to provide a reference within the three-dimensional model that may be used for registering the model to the reference frame of patient anatomy during subsequent procedures while the implant state may be used to update the three-dimensional model as elaborated on below.

In one embodiment of the above, an active implant may be delivered and anchored during an initial procedure. In one example, a robotic or manual system including a localized instrument (such as a robotic endoluminal platform or a manually actuated instrument with position sensing) can be used to perform a biopsy on a target tissue segment, or other medical procedure, and an active implant can be delivered using the localized instrument to a desired location within the subject to facilitate registration between a model of the subject's anatomy captured during an initial procedure and the anatomy of the subject during subsequent procedures. For instance, the active implant may be delivered to a location corresponding to the closest carina proximal to the target tissue segment. In some embodiments, a three-dimensional model may be updated based on a determined positional information of the active implant within the subject.

The positional information of the active implant may be determined in a number of ways. For example, in one embodiment, the location and orientation of the active implant may be determined based on signals from localization sensors of a delivery instrument registered to the three-dimensional model that are capable of sensing a location and orientation of at least a distal end portion of the instrument from which an active implant may be deployed. Specifically, due to the delivery instrument being localized (e.g., the instrument includes sensors such as shape, electromagnetic sensors, and/or other appropriate sensors configured to determine a real-time position and orientation of at least a distal portion of the instrument), the location and orientation (i.e. a pose) of the active implant may be known relative to the instrument when it is deployed at a desired location. Implant state information (e.g. initial relative state of the active implant fixed within patient anatomy used to measure a change in scale, translation, bending, and rotation) of the active implant may be known based on known relative locations of multiple sensors/fiducials coupled or fixed to the active implant and/or determining location and orientation of multiple active implants relative to one another.

In another example, the clinician may interact with the anatomical model and identify the location, orientation, or implant state information of the implant within the model. However, regardless of the method used to determine positional information of the active implant, the positional information of the active implant may then be used to correlate the anatomic model with patient anatomy and optionally update the anatomic model for usage during either the same or subsequent procedures.

As noted above, in some embodiments, a subsequent follow-up medical procedure may be performed at some time after an initial medical procedure during which an active implant may be deployed into the anatomy of a subject. This follow-up medical procedure can be performed with a robotic or manual system using the three-dimensional model generated during the initial medical procedure. The inventor has recognized and appreciated that because the active implant is anchored at a known location with a known orientation (e.g. a known pose) within the subject anatomy, a sensed pose of the active implant may facilitate the identification of target tissue and/or the registration of the model with the reference frame of a patient anatomy on which a subsequent medical procedure is being performed. For example, the relative positioning between the closest carina an active implant is deployed at and a target tissue such as a lesion may be consistent from the initial procedure to the follow-up procedure. Thus, using the pose of the active implant to register the three-dimensional anatomical model with the reference frame of the patient anatomy may provide an accurate location of the target tissue and other regions of interest within the model. Additionally, by registering an instrument being used during the follow-up procedure with the active implant, real time guidance within the anatomical model may be provided showing a relative location of the instrument to the target tissue.

In some embodiments, the subject anatomy can change, shift, or deform between the initial medical procedure and the follow up medical procedure. Accordingly, the active implant which is fixedly attached to anatomy can deform in a similar manner. By measuring the deformation of the active implant by comparing the implant scale information recorded during an initial procedure with implant scale information determined during the follow up procedure, a similar deformation can be applied to the anatomic model that reflects a real deformation of anatomy. For example. during the initial procedure, after the implant has been deployed within the anatomical structure, a baseline implant state of the active implant may be taken. For example, as anatomy deforms, the active implant will stretch or compress, bend, and twist. If the relative location and orientation of fixed structures, e.g. multiple sensors fixed to the active implant can be determined during the initial procedure and saved as a baseline, then the relative location and orientation of the fixed structures may be measured or determined during a follow up procedure and compared to the baseline measurements to determine deformation of the active implant which can be used to determine deformation of the anatomic model.

In one embodiment, a method for providing guidance during a follow-up procedure may include using a previously implanted active implant disposed within subject anatomy and a three-dimensional model including a reference within the model corresponding to a previously determined location and orientation of the active implant. This three-dimensional model may be obtained in any appropriate manner including recall from memory, transfer from memory device, downloading from a remote server, and/or any other appropriate method. In addition, positional information of the active implant within the subject's anatomy may be determined during the follow up procedure. For example, a location and orientation (i.e. pose) of the active implant may be sensed by one or more therapeutic localizations sensors associated with an instrument used to perform the desired medical procedure as elaborated on below. The anatomical model and determined pose of the active implant may then be used to register the model to a reference frame of the instrument based on the pose of the active implant.

The inventor has recognized and appreciated that the disclosed active implants and the related methods of use may reduce errors associated with subject and/or anatomical movements (e.g., lung inflation, deflation, rotation, stress, strain, distortion, etc.), such as by tracking and registration of the active implant and the anatomical model with the reference frame of the patient anatomy and optionally, an instrument in real-time. This may lead to less medical complications given the more accurate, subject-specific, and continually reliable localization of an instrument during a procedure. Additionally, in some instances the disclosed methods and system may provide more confidence in subject-specific anatomies, guiding more efficient intraprocedural decision making, such as via leveraging a pre-operative plan to guide a follow-up medical procedure using the registration provided by the disclosed active implants. In other embodiments, the inventor has also recognized that additional imaging may not be needed when employing systems and techniques herein in some instances, as a user can drive toward the target more confidently than with other conventional systems which registration between a model and a reference frame of an instrument being operated is not as well known.

While the disclosed systems and methods may be used for any number of applications, in some embodiments, the disclosed techniques may enable the easy and repeated access to a region of interest either during a single operation including multiple procedures and/or during procedures that are spaced apart in time. For example, the disclosed techniques may enable endoluminal-accessed serial drug/biologic delivery to a tumor, as well as ablation using RF, microwave, cryotherapy, ultrasound therapy, laser, direct heat delivery, and/or other therapeutic procedures during sequential operations done using either the same or different instruments. The inventor has recognized and appreciated that the techniques disclosed herein may be helpful for treatment of lungs, including segmentectomies and wedge resection (e.g., marking the location of a tumor, finding the closest carina to the desired anatomical location, etc.), the treatment of the gastrointestinal tract, biliary duct, and/or any other lumen or appropriate anatomical structure in the body using any appropriate type of treatment as elaborated on below.

The inventor has recognized and appreciated that embodiments disclosed herein may provide improved localization that may also be helpful in other situations in addition to the above. For example, the improved localization may be helpful for removal of the implant if the target area is found out to not be diseased (e.g., not cancerous). Alternatively or additionally, the improved localization may be helpful for removal of the implant after treatment (such as during a follow-up procedure).

Embodiments disclosed herein may be used with any medical instrument. This may include robotic or manually operated endoscopes, catheters, and rigid instruments as well as manually operated systems, robotic assisted surgical systems, teleoperated robotic surgical systems, and/or other desired applications. Of course, it should be understood that the disclosed techniques are not limited to use with only these specific applications as the disclosure is not so limited.

As used herein, the term “position” refers to the location of an element or a portion of an element in a three-dimensional space (e.g., three degrees of translational freedom along cartesian x- , y- , and z-coordinates). As used herein, the term “orientation” refers to the rotational placement of an element or a portion of an element (three degrees of rotational freedom—e.g., roll, pitch, and yaw, angle-axis, rotation matrix, quaternion representation, and/or the like). As used herein, the term “pose” refers to the multi-degree of freedom (DOF) spatial position and orientation of a coordinate system of interest attached to a rigid body. In general, a pose includes a pose variable for each of the DOFs in the pose. For example, a full 6-DOF pose would include 6 pose variables corresponding to the 3 positional DOFs (e.g., x, y, and z) and the 3 orientational DOFs (e.g., roll, pitch, and yaw). In view of the above, it should be understood that a pose may be defined as a combination of a position and orientation of a body. Pose information fully defines the spatial state of a body in a coordinate system reference frame. For example, both an individual body and a kinematic chain of bodies may each have an associated pose, and it may be important to distinguish the meaning of pose in this context.

Turning to the figures, specific non-limiting examples are described in further detail. It should be understood that the various systems, components, features, and methods described relative to these examples may be used either individually and/or in any desired combination as the disclosure is not limited to only the specific examples described herein.

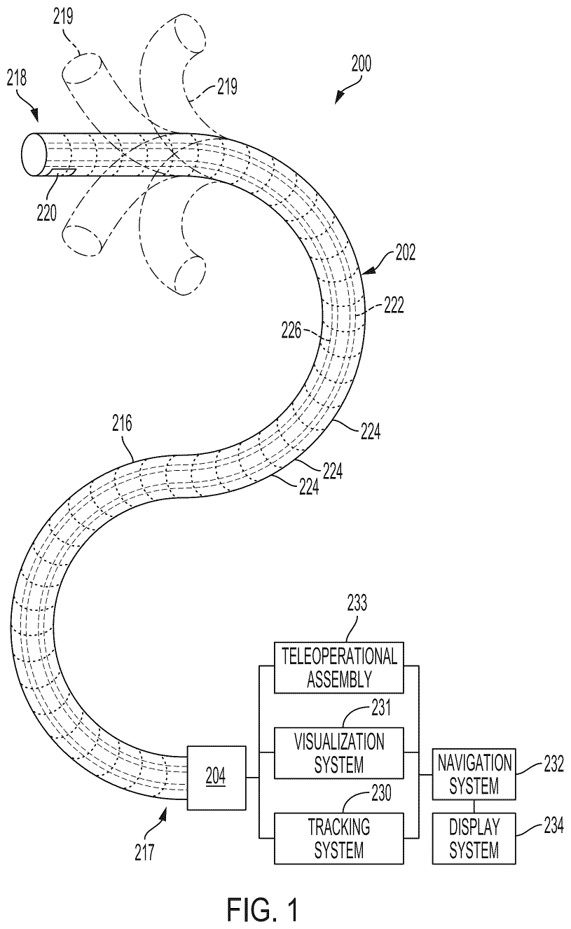

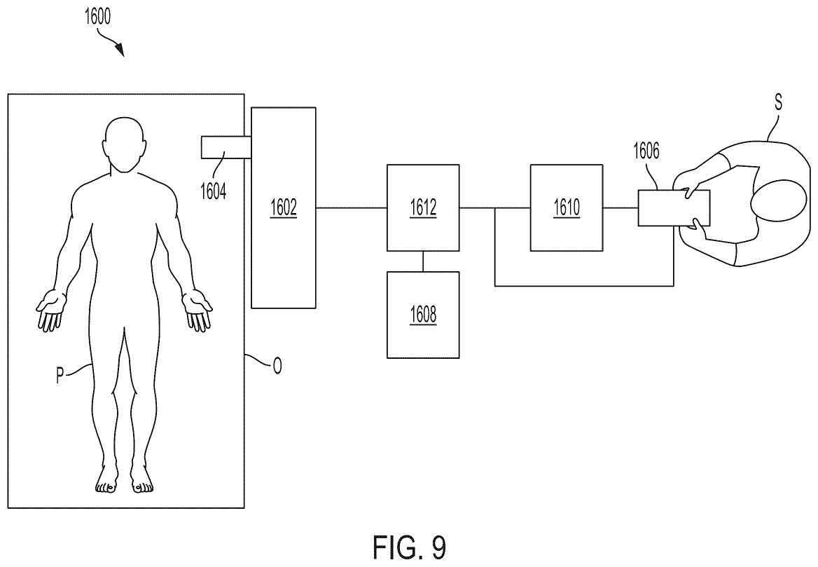

FIG. 1 illustrates an instrument system 200, which may be used as a medical instrument system in an image-guided medical procedure performed with a teleoperational medical system (e.g., 1600 of FIG. 9) using teleoperational assembly 233. Alternatively, the medical instrument system 200 may be used for non-teleoperational exploratory procedures or in procedures involving traditional manually operated medical instruments, such as endoscopy. Additionally or alternatively the medical instrument system 200 may be used to gather (i.e., measure) a set of data points corresponding to locations with subject anatomic passageways.

The instrument system 200 includes a catheter system 202 coupled to an instrument body 204. The catheter system 202 includes an elongated flexible catheter body 216 having a proximal end 216 and a distal end or tip portion 218. The catheter system 202 may optionally include a localization sensor (e.g. a shape sensor 222, EM sensor 220, and/or series of EM sensors) for determining the position, orientation, speed, velocity, pose, and/or shape of the catheter tip, or other distal end portion, at distal end 218 and/or of one or more segments 224 along the body 216. If the instrument system 200 is a medical instrument system (e.g., 1604) of a teleoperational medical system (e.g., 1600), the localization sensor may be a component of the sensor system 108. If the instrument system 200 is manually operated or otherwise used for non-teleoperational procedures, the localization sensor may be coupled to a tracking system 230 that receives the sensor data and processes the received data to determine position and optionally orientation of a distal end, section, or entire length of the instrument.

The localization sensor may include shape sensor 222 may include an optical fiber aligned with the flexible catheter body 216 (e.g., provided within an interior channel (not shown) or mounted externally). Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fibre Bend Sensor”), which are all incorporated by reference herein in their entireties. Additionally or alternatively, the localization sensor may include a positional sensor or a series of positional sensors such as EM sensors. In some examples, the EM sensors may be positioned along the catheter for shape sensing. This type of EM sensor may additionally be used with the active implants as described previously above. Further description of an EM sensor system is provided in U.S. Pat. No. 6,380,732 (filed Aug. 11, 1999) (disclosing “Six-Degree of Freedom Tracking System Having a Passive Transponder on the Object Being Tracked”), which is incorporated by reference herein in its entirety. In some embodiments, the shape sensor may also function as the position sensor because the shape of the sensor together with information about the location of the base of the shape sensor (in the fixed coordinate system of the instrument) allows the location of various points along the shape sensor, including the distal tip, to be calculated.

A tracking system 230 may include the position sensor system 220 and/or a shape sensor system 222 for determining the position, orientation, speed, pose, and/or shape of the distal end 218 and of one or more segments 224 along the instrument 200. The tracking system 230 may be implemented as hardware, firmware, software or a combination thereof which interact with or are otherwise executed by one or more computer processors, which may include the processors of a control system.

The medical instrument 226 may house cables, linkages, or other actuation controls (not shown) that extend between the proximal and distal ends of the instrument to controllably bend the distal end of the instrument. Steerable instruments are described in detail in U.S. Pat. No. 7,316,681 (filed on Oct. 4, 2005) (disclosing “Articulated Surgical Instrument for Performing Minimally Invasive Surgery with Enhanced Dexterity and Sensitivity”) and U.S. patent application Ser. No. 12/286,644 (filed Sep. 30, 2008) (disclosing “Passive Preload and Capstan Drive for Surgical Instruments”), which are incorporated by reference herein in their entireties.

In embodiments in which the instrument system 200 is actuated by a teleoperational assembly, the housing 204 may include drive inputs that removably couple to and receive power from motorized drive elements of the teleoperational assembly. In embodiments in which the instrument system 200 is manually operated, the housing 204 may include gripping features, manual actuators, or other components for manually controlling the motion of the instrument system. The catheter system may be steerable or, alternatively, the system may be non-steerable with no integrated mechanism for operator control of the instrument bending. Also or alternatively, one or more lumens, through which medical instruments can be deployed and used at a target surgical location, are defined in the walls of the flexible body 216. For example, a channel maybe be sized and shaped to receive a medical instrument such as image capture probes, biopsy instruments, laser ablation fibers, or other surgical, diagnostic, or therapeutic tools, or an active implant.

The information from the tracking system 230 may be sent to a navigation system 232 where it is combined with information from the visualization system 231 and/or the preoperatively obtained models to provide the surgeon or other operator with real-time position information on a display system (e.g., 1610 in FIG. 15) for use in the control of the instrument 200. The control system may utilize the position information as feedback for positioning the instrument 200. Various systems for using fiber optic sensors to register and display a surgical instrument with surgical images are provided in U.S. patent application Ser. No. 13/107,562, filed May 13, 2011, disclosing, “Medical System Providing Dynamic Registration of a Model of an Anatomical Structure for Image-Guided Surgery,” which is incorporated by reference herein in its entirety.

In the embodiment of FIG. 1, the instrument 200 is teleoperated within a telcoperational medical system (e.g., 1600 in FIG. 9). In an alternative embodiment, the teleoperational assembly (e.g., 1602 in FIG. 9) may be replaced by direct operator control. In the direct operation alternative, various handles and operator interfaces may be included for hand-held operation of the instrument.

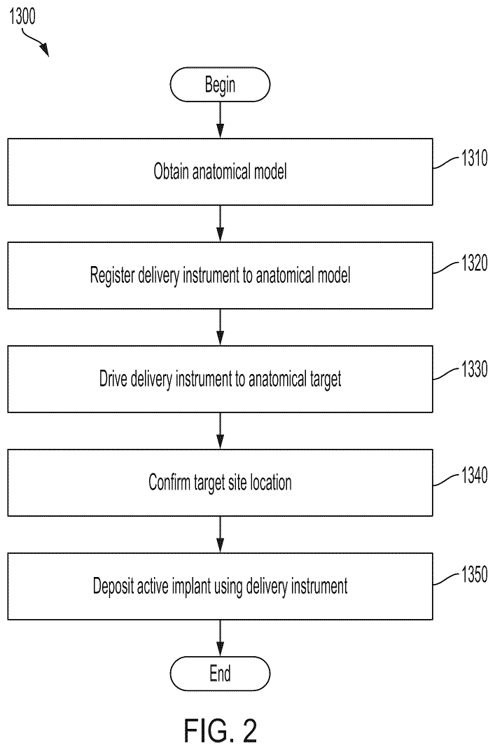

FIG. 2 is a flowchart illustrating one embodiment of an initial procedure during which an active implant may be implanted within a subject for facilitating the registration of an anatomical model with the reference frame of an instrument. In the depicted embodiment, the process 1300 is illustrated in FIG. 2 as a set of stages, blocks, steps, operations, or processes. Not all of the illustrated, enumerated operations may be performed in all embodiments of the process 1300. Additionally, some additional operations that are not expressly illustrated in FIG. 2 may be included before, after, in between, or as part of the enumerated stages.

Some embodiments of the process 1300 may begin at stage 1310 where an anatomical model of the subject may be obtained. In one such embodiment, the anatomy of a subject may be imaged either before or during an operation with any appropriate instrument to develop an anatomical model of at least a portion of the subject. In this embodiment pre-operative or intra-operative image data can be obtained from imaging technology such as, computed tomography (CT), magnetic resonance imaging (MRI), fluoroscopy, thermography, ultrasound, optical coherence tomography (OCT), thermal imaging, impedance imaging, laser imaging, or nanotube X-ray imaging. Computer software alone or in combination with manual input can then be used to convert the recorded images into a segmented two dimensional or three-dimensional composite representation or model of a partial or an entire anatomical organ or anatomical region. The composite representation and the image data set describe the various locations and shapes of anatomy such as airway passageways and their connectivity.

A processor of the system may be configured to receive user input to determine or automatically determine an anatomical target and optionally generate a planned path to the anatomical target in the anatomical model. The processor may be configured to provide guidance, and/or may control the delivery instrument, to navigate the delivery instrument to the anatomical target based on the planned path. The processor may be configured to identify or receive information identifying a location for deployment of the active implant. For example, a location proximate to the anatomical target may be identified either automatically, semi-automatically (e.g., a system determines and recommends a location), or by a user. In cases in which the location is selected either automatically or semi-automatically, a processor of the system may be configured to identify these locations based on appropriate considerations such as relevant landmark and anatomical structures within the anatomical model that are proximate to the anatomical target. This may include in some embodiments the next proximally located carina relative to the anatomical target for a procedure conducted in the lungs of a subject.

After obtaining the anatomical model, the process 1300 may then proceed to stage 1320, in which a delivery instrument (such as medical instrument system 200) and subject anatomy may be registered to the three-dimensional anatomical model of the subject anatomy. Generally, registration involves the matching of measured points to points of the model through the use of rigid and/or non-rigid transforms. Measured points may be generated using landmarks in the anatomy, electromagnetic coils scanned and tracked during the procedure, or a shape sensor system. This registration may be done using any appropriate technique including, but not limited to, landmark identification, point cloud comparisons, and/or other appropriate techniques. Other point set registration methods may also be used in registration processes within the scope of this disclosure. Additionally, in some instances, the anatomical model may be registered to a reference frame of the instrument due to the anatomical model being generated by the instrument during the current procedure.

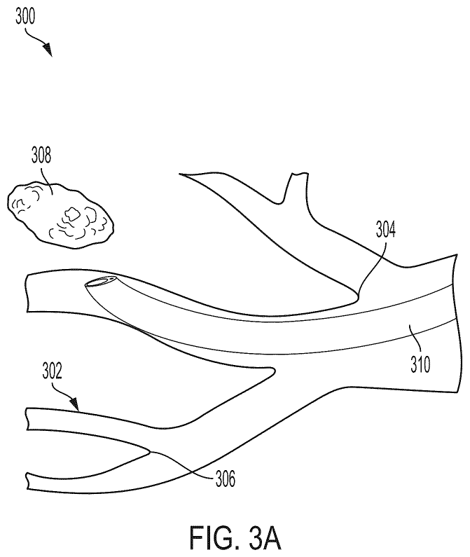

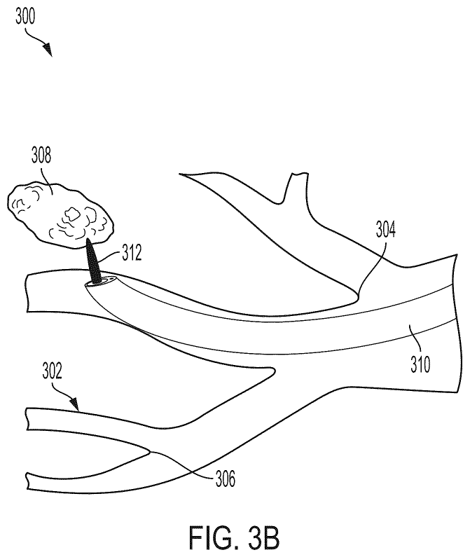

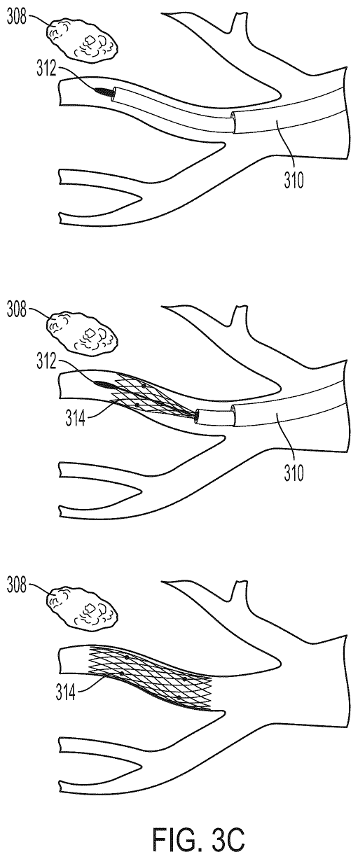

After registering the anatomical model to a reference frame of the instrument, method 1300 may then proceed to stage 1330, in which the delivery instrument may be driven to a location of the anatomical target as illustrated in FIG. 3A. FIG. 3A illustrates an exemplary bronchial anatomical model 300 showing the different anatomical structures, a target segment of the anatomy, and exemplary systems, in accordance with embodiments of the present disclosure. Specifically, the figure depicts anatomical passageways 302 as well as main carinas 304 and carinas 306 at junctions between different branches along the illustrated passageways. The delivery instrument 310 may be driven to the anatomical target 308 using any appropriate method. For example, a user may manually control the delivery instrument, use the anatomical model for guidance, or a pre-planned path may be implemented, to guide the delivery instrument to the anatomical target.

Once the delivery instrument 310 is appropriately positioned proximate to the anatomical target 308, a target site location of the anatomical target may be confirmed at 1340 using any appropriate method as illustrated in FIG. 3B. This may include, for example: a biopsy needle 312, or other instrument, delivered through the delivery instrument being used to obtain a sample of the anatomical target 308; visual identification of the anatomical target 308 using an endoscopic camera; imaging of the anatomical target 308 using an ultrasound device delivered through or integrated within the delivery instrument; and/or any other appropriate procedure capable of confirming the anatomical target 308.

After the target site location has been confirmed, method 1300 may then proceed to stage 1350, in which the delivery instrument 310 may be used to deposit an active implant 314 at a desired location and orientation (i.e. pose) within the anatomical structure of the subject as illustrated in FIG. 3C and as will be described in further detail below. In some examples, the desired location is determined during a planning stage such as during stage 1310. In other examples, the desired location may be determined after confirming the target site location and selecting a location proximate to the current position of the delivery instrument at the anatomical target.

In some embodiments, after depositing an active implant in a desired pose relative to one or more anatomical structures of a subject, it may be desirable to update an anatomical model, such as a three-dimensional anatomical model, of the subject to include a reference related to the positional information of the active implant within the subject. The positional information may include a location and orientation (pose) of the implant within the subject anatomy and implant state information which can be used to determine an initial relative state of the active implant fixed within patient anatomy used to measure a change in scale, translation, bending, and rotation. As noted previously, the information, which may include a pose, of the implant within anatomy may facilitate the use of the model in subsequent procedures through the ability to sense a pose of the active implant. The implant state information may be used to update the model during subsequent procedures to account for changes and shifts in a subject anatomy after the initial medical procedure.

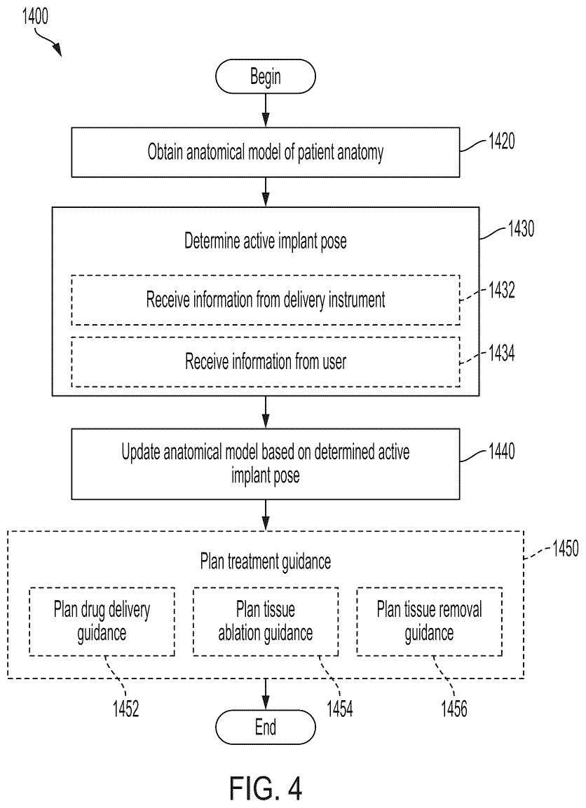

Such a method is shown in FIG. 4 which is a flowchart of one embodiment illustrating a method 1400 used to update an anatomical model of a subject with the positional information of an active implant within a portion of a subject's anatomy, and in some instances, optionally to plan guidance for a clinician to use in an image-guided surgical procedure on the subject. The method 1400 is illustrated in FIG. 4 as a set of stages, blocks, steps, operations, or processes. Not all of the illustrated, enumerated operations may be performed in all embodiments of the method 1400. Additionally, some additional operations that are not expressly illustrated in FIG. 4 may be included before, after, in between, or as part of the enumerated stages. Some embodiments of the method 1400 include instructions corresponding to the processes of the method 1400 as stored in a memory. These instructions may be executed by a processor like a processor of a control system.

At 1420, an anatomical model such as the anatomical model generated during method 1300 of FIG. 2, may be obtained. After obtaining the anatomical model, positional information of the active implant within the subject may be determined at 1430. The positional information may include a location and orientation (pose) of the implant within the subject anatomy and implant state information which can be used to later determine a change in the structure of the active implant to be used to determine a change in the anatomy in which the active implant is fixed. Include. Obtaining the positional information may either be done in real time using a system that delivers the active implant or it may be performed by a separate system using previously recorded information as the disclosure is not so limited. The positional information of the active implant relative to the tissue it is implanted into within the subject's anatomy may be determined in a number of different ways including receiving information from a delivery instrument at 1432, using known active implant sensor structural information, using imaging techniques, and/or information from a user at 1434 as elaborated on below.

In one embodiment, at 1432 a delivery instrument localization sensor such as a shape sensor, EM sensor, plurality of EM sensors, or other appropriate sensor may be used to determine a pose of a distal portion of the delivery instrument from which the active implant is deployed. In instances in which the anatomical model is registered to the reference frame of the delivery instrument, the pose of the distal portion of the delivery instrument may be used to determine a pose of the active implant relative to a reference frame of the model once the active implant has been deployed, i.e., a delivery pose of the active implant as measured by the delivery instrument localization sensor may be used.

In another embodiment, the active implant may include multiple localization which are each embedded, coupled or otherwise fixed to the active implant in a known mechanical configuration which can be used to determine implant state information. In one example, the relative distances between the multiple localization sensors including axial and rotational distance along the active implant can be recorded as positional information at the time of deployment within the subject anatomy. In another example, the relative distances of each of the localization sensors may be measured. In further examples, the localization sensors may be used as fiducials which are visible in images and the relative distances may be visualized (e.g. visualizing under fluoroscopy used during the implanting procedure or internal imaging provided by the instrument) and processed via image processing or identified visually by users.

In yet another embodiment, the implant state information may be determined using multiple active implants, each including one or more localization sensor. In this embodiment, the method would include delivering multiple active implants in close proximity to one another within the subject anatomy, using the delivery instrument with localization sensing (EM, fiber shape, etc.) as previously described. The relative distances may be determined between each of the active implants by use of data from a delivery instrument with localization sensing, measurement of signals from each of the implant localization sensors, and/or use of imaging to visualize the implant localization sensors as fiducials, as described above.

In another embodiment, at 1434 information related to the pose of the active implant may be input by a user using any appropriate input device. For example, a user may use a graphical user interface to select a pose within the model corresponding to the location of a deployed active implant as well as select where localization sensors on the active implant are located. In a further embodiment, both sensor data from the delivery instrument and user input can be used to determine the active implant pose with sensor data being initially used and user input provided to adjust the pose or user input provided for an initial pose and sensor data used to refine the position or orientation within the model.

While specific methods for determining the pose of an active implant are described above, it should be understood that any appropriate method for determining the pose of an active implant may be used as the disclosure is not so limited.

After determining the pose of an active implant relative to the reference frame of the anatomical model and the implant state of the active implant, the method may proceed to stage 1440 where the anatomical model may be updated based on the determined positional information of the active implant. Specifically, a reference corresponding to the pose and implant state of the implant within the model may be incorporated into the model. Accordingly, the pose and implant state of this reference in the model may substantially correspond to a pose and implant state of the active implant within the corresponding anatomical structure of the subject.

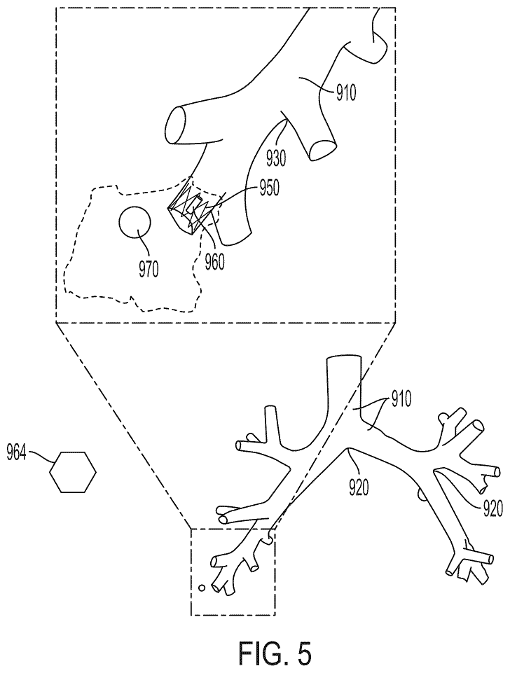

FIG. 5 illustrates an exemplary bronchial anatomical model showing the different anatomical structures, a target segment of the anatomy, and exemplary systems, in accordance with embodiments of the present disclosure. Specifically, the figure depicts anatomical passageways 910 as well as main carinas 920 and carinas 930 at junctions between different branches along the illustrated passageways. As described previously, an active implant 960 may be implanted at a desired location within the patient anatomical structure with a given orientation resulting in an overall pose of the active implant within the anatomical structure. As previously described, with a known position and orientation within the anatomical structure (known from delivery of the active implant with a registered delivery instrument or by user input), the position and orientation within the anatomical model is known and a virtual representation of the active implant can be added to the anatomical model.

In some embodiments, it may be desirable to provide treatment guidance for a user, such as a medical practitioner, after a model including information related to an active implant has been generated. For example, in some embodiments, method 1400 may optionally proceed to stage 1450, in which treatment guidance may be planned. Depending on the embodiment, the treatment guidance may correspond to providing drug delivery guidance at 1452, tissue ablation guidance at 1454, tissue removal guidance at 1456, guidance for sequential treatments, combinations of the foregoing and/or any other appropriate type of treatment guidance.

In one embodiment of stage 1450, a processor may be configured to receive the three-dimensional model including an anatomical target and the information related to the active implant pose. A section of the subject anatomy for treatment based on the three-dimensional model may be identified. This may be an automatic step conducted by the system where locations for stapling, cutting, or other treatment are identified. Alternatively, the system may implement this step semi-automatically where the system defines the locations and types of treatment, but then the user can change the suggested treatment. In yet another embodiment, a user may manually enter the plan in a pre-planning stage performed before the follow up procedure. It should be understood that any medical instrument system used to implement the planned treatments may have an appropriate therapeutic device to perform removal of an identified anatomical target, deliver therapeutics such as drugs to the identified anatomical target, and/or to provide any other desired type of treatment to the identified anatomical target as the disclosure is not limited.

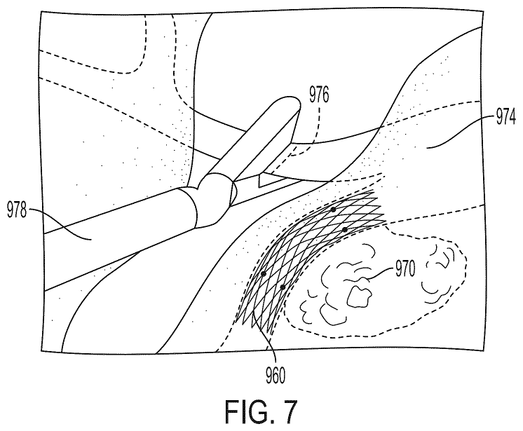

FIGS. 7 illustrates an example of graphical guidance using an active implant 960 in a human body, in accordance with embodiments of the present disclosure. FIG. 7 depicts an exemplary target tissue segment 970 within a lumen of an anatomical space. In the depicted example, the anatomical space corresponds to a lung of a subject. While the target tissue segment is visible within the field of view of the instrument, it may still be difficult to identify other surrounding tissue. However, if an anatomical model of the subject is appropriately registered to a reference frame of the instrument, it may be possible to display additional information to a user. For example, as illustrated in FIG. 7 information such as the locations of one or more identified anatomical structures, including the target tissue segment (i.e., an anatomical target), may be added to the anatomical model. Specifically, FIG. 7 depicts the exemplary target tissue segment 970 along with other structures such as an identified vein (not depicted) and bronchi structures 974 as well as recommended surgical cut or suture lines 976. The surgical cut/suture lines 976 can be added by the user through a graphical user interface or automatically determined by the system. A location of an instrument 978 within the registered anatomical model, and a corresponding location within the anatomical space, may be superimposed on an image presented to the user in some embodiments. In another embodiments, the additional information may be overlaid on live fluoroscopic or endoscopic images after registration of the imaging devices to the anatomical model. Thus, in some embodiments, the disclosed systems and methods may be used to facilitate augmented reality visualization during a surgical procedure which may accelerate procedure speeds and success rates.

It should be noted that the above described processes for the deployment and updating of a model may be repeat as many times as needed if multiple active implants are to be delivered into the anatomy of a subject.

As noted previously, an active implant (or multiple active implants) disposed within a subject during an initial procedure may be used to facilitate the registration between a previously generated model of the subject anatomy correlated with the active implant and the same subject anatomy during a later follow-up procedure. One such embodiment is shown in FIG. 13 which is a flowchart illustrating an exemplary process 1500 of using exemplary systems in a follow-up procedure after a model including a reference to positional information of the active implant has been generated. The method is illustrated as a set of stages, blocks, steps, operations, or processes. Not all of the illustrated, enumerated operations may be performed in all embodiments of the method 1500. Additionally, some additional operations that are not expressly illustrated in FIG. 13 may be included before, after, in between, or as part of the enumerated stages. Some embodiments of the method 1500 include instructions corresponding to the processes of the method 1500 as stored in a memory. These instructions may be executed by a processor like a processor of a control system disclosed herein.

Some embodiments of the process 1500 may begin at stage 1510 in which the medical instrument system may obtain an anatomical model of the patient anatomy, such as the anatomical model generated during method 1440 of FIG. 4, for the follow up procedure to be conducted. At 1520, positional information of the active implant(s) within the current patient anatomy may be determined. As previously described, the positional information can include active implant pose within anatomy and implant state information. The pose is known in an implant localization sensor reference frame. Since the active implant is fixed and imbedded within the subject anatomy, the implant localization reference frame can be considered the same as an anatomical reference frame of the subject. In some embodiments, the desired positional information includes a state of the active implant, including relative positioning of each of the implant localization sensors 962. In this embodiment, the active implant should be at least partially flexible and have a longitudinal length which can provide for an adequate separation of multiple spaced sensors. The state of the active implant can include the relative positioning of the implant localization sensors to one another including the distance between each sensor along a longitudinal axis of the implant, a radial distance along a radius of the active implant, and/or a rotational distance between each sensor. Several potential embodiments for detecting the positional information of the active implant are detailed further below.

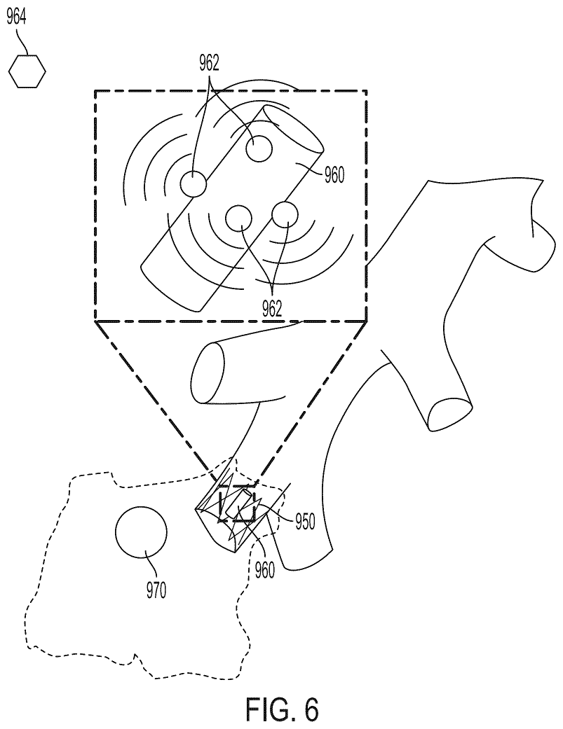

As best seen in FIG. 6, the active implant 960 may include one or more implant localization sensors 962 in some embodiments. It should be understood that any appropriate type of active implant may be used that is capable of measuring position information. For example, the implant localization sensor may either be a passive system and/or it may be powered by an onboard power source (e.g., a battery) or it may be powered inductively by an externally located power source. In the case of a passive active implant, the active implant may include inductive circuits with one or more inductive coils configured to be coupled with electromagnetic fields emitted by two or more, three or more, or any other appropriate numbers of receivers (e.g., corresponding therapeutic localization sensors) configured to be disposed at different locations relative to the active implant such that the sensed signals may be used to determine a pose of the active implant. In some embodiments, multiple inductive circuits may be included in an active implant where the different inductive circuits may be configured to be excited at different frequencies and or with different modulation frequencies. In other embodiments, radiopaque fiducials included in an active implant may be imaged by an external X-ray imaging system to determine a pose of the active implant. In the case of a powered active implant, in some embodiments, the implant localization sensor may include one or more transmitters configured to emit a pulsed signal that may be sensed by two or more, three or more, or any other appropriate number of receivers of a separate instrument configured to sense the emitted pulsed signals which may then be used to determine a pose of the active implant. Of course, other types of active implants with either passive and/or active components capable of being used to detect positional information related to the active implant may be used as the disclosure is not so limited.

Depending on the specific application, an active implant may be anchored relative to a target location within a subject's anatomy in any appropriate fashion. For example, in some embodiment, appropriate attachment types may include, but are not limited to, stents, clips, adhesives, coiled elements, baskets, and/or any other appropriate method for attaching the active implant to an adjacent portion of tissue. As illustrated in FIG. 3C, the active implant 314 may be an expandable stent delivered and deployed using a delivery instrument 310.

In some embodiments, the active implant may be hermetically sealed or sealed in any other suitable way for placement inside a human body. The active implant may be held in the desired pose within the anatomical structure using any appropriate attachment method as noted previously. For example, the illustrated stent 950 is engaged with the surrounding tissue of the passageway of the lungs such that a pose of the stent, and correspondingly a pose of the active implant connected to the stent, is maintained relative to the surrounding tissue. A target tissue segment 970 that is spaced from a location of the active implant is also depicted.

In embodiments in which a powered active implant is used, the active implant may either be powered by an onboard power source such as a battery or the active implant may be powered inductively through one or more externally located power sources (e.g., an inductive power source included in the medical instrument system 200 or the teleoperated system 1600). In either case, one or more implant localization sensors of an active implant deposited in a previous procedure may correspond to one or more transmitters configured to transmit a wireless signal such as one or more characteristic radiofrequencies or modulations on a shared frequency to enable differentiation between elements (such as multiple implants). The sensed transmissions emitted by the active implant may be used to determine the pose of the active implant relative to the sensors, and thus a reference frame, of the associated system medical instrument system used to perform the desired medical procedure.

In another embodiment, an active implant may be at least partially radiopaque and have physical characteristics like size, shape, identity of marker, distance between elements, and/or any other characteristics that may be used to identify both a location and orientation of the active implant using multiple X-ray images (e.g., at least 2) taken at different poses relative to the subject. Accordingly, the X-ray images may be used to identify a pose of the active implant within the subject. Additionally, in instances in which a reference frame of the X-ray imager is registered with the reference frame of the medical instrument system, the pose of the active implant relative to the medical instrument system may be determined.

While specific methods for determining a pose of an active implant implanted within the anatomy of a subject are detailed above, it should be understood that any appropriate method of determining the pose of an active implant relative to the reference frame of a medical instrument system used to perform one or more medical procedures on the subject may be used as the disclosure is not limited in this fashion.

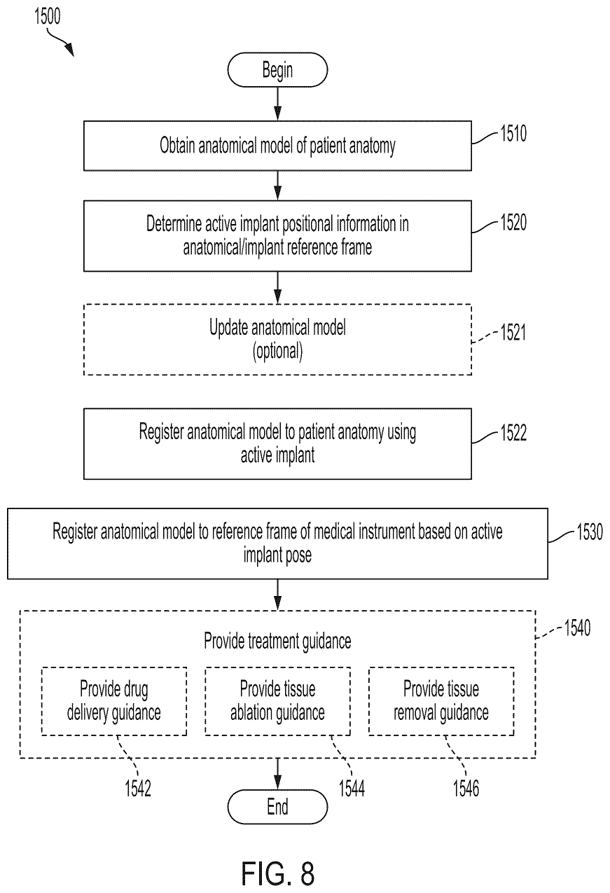

Referring back to method 1500 of FIG. 8, at stage 1521, the anatomical model may optionally be updated. In some embodiments, the subject anatomy can deform between the initial medical procedure when the active implant is deployed and the follow up medical procedure. Accordingly, the active implant which is fixedly attached to anatomy can deform in a similar manner. By measuring the deformation in the active implant from the initial procedure to the follow up procedure a similar deformation can be applied to the anatomic model that reflects a real deformation of anatomy. Since the anatomical model obtained during stage 1510 includes implant state information, providing for a baseline measurement of the initial relative positions of the implant localization sensors 962, the implant state information obtained from stage 1520 can be used to determine a deformation of the active implant. For example, measuring a change longitudinal or radial distance can measure a change in scale indicating a stretching, compression, or bending of the implant, a rotational change can indicate a twist of the implant, etc. The changes measured in the active implant can then be translated to changes in the anatomical model, and the anatomical model can be updated to reflect this deformation. In another embodiment with multiple active implants, the change in relative distances between each sensor can be determined in a similar manner with the exception that the sensors are imbedded in/coupled to separate active implants which are deployed in different locations in the subject anatomy.

In yet another embodiment, an instrument used during a follow up procedure may include an imaging device which can be used to provide additional information to determine anatomical deformation. For example, an instrument with multiple endoscopic cameras can provide stereoscopic vision which can be used to extract surface features within a subject anatomy that can be treated as boundary conditions to deform the anatomical model to more accurately reflect the anatomy at the time of the follow up procedure. By additionally visualizing the active implant/s, the expected location of the fiducials can be compared with the actual location of the fiducials to further update the model. Imaging analysis or artificial intelligence may be used to identify particular structures within the video stream or a user may annotate a video as it is being displayed using a GUI or other interactive input device.

After determining the pose of an active implant and optionally updating the anatomical model, the process 1500 may then proceed to stage 1522, in which the anatomical model is registered to the subject anatomy using the active implant. As previously described, the anatomical model from method 1440 includes positional information for the active implant including the pose of the active implant within the model. Thus, the known pose of the active implant within the anatomical model can be used to register the anatomical model to the subject anatomy using the measured pose of the active implant obtained from stage 1520.

After registering the three-dimensional anatomical model to the patient anatomy, the process 1500 may then proceed to stage 1530, in which the three-dimensional anatomical model of the subject anatomy may be registered to a reference frame of a medical instrument system, or other medical platform that can be used to perform a medical procedure on the subject anatomy (e.g. tumor removal, segmentectomy, wedge resection, ablation, drug delivery, radiotherapy treatment, lymph tract identification, etc.). For example, as noted previously the anatomical model may include a reference within the model related to the pose of the active implant relative to the anatomical model. Thus, the anatomical model may be registered to a reference frame of the medical instrument system which can include one or more therapeutic localization sensors, based at least partly on registration of the active implant sensor reference frame to the medical instrument system therapeutic sensor reference frame. Several potential embodiments for registration of the implant localization sensors to the therapeutic localization sensors are detailed further below.

In one embodiment, the one or more implant localization sensors may be configured such that they may either actively or passively be sensed by one or more corresponding therapeutic localization sensors 964 configured to sense information related to a pose of the active implant. The therapeutic localization sensors may either be included in a medical instrument system and/or may be registered to a reference frame of the medical instrument system and may be disposed externally to the subject.

In yet another embodiment, the implant localization sensors and the corresponding therapeutic localization sensors may function as an EM sensor which is described in more detail above. In such an embodiment, the implant localization sensors may correspond to one or more circuits including inductive coils that may be coupled with electromagnetic fields emitted by one or more magnetic field emitters disposed outside of the subject (i.e., the one or more therapeutic localization sensors of the medical instrument system). As described previously, the interactions between the emitted electromagnetic field(s) and the inductive circuits of the active implant may be used to determine a pose of the active implant. In some embodiments, a reference frame of the therapeutic localization used to measure the pose of the active implant may be the same as the reference frame the medical instrument system. Accordingly, the pose of the active implant measured with the therapeutic localization sensors may correspond to the pose of the active implant relative to the medical instrument system.

In another embodiment, the medical instrument can be used to touch portions of the active implant and the location and orientation of the medical instrument tip or distal portion of the medical instrument can be measured and correlated with known positions on the active implant. Touching multiple know points on the active implant can provide data required to register the active implant reference frame to the medical instrument reference frame.

It should be understood that stages 1520 through 1530 corresponding to the detection of the positional information of an active implant and registration of the anatomical model to a reference frame of a subject anatomy and a medical instrument system may be performed a single time, continuously in real time to account for movement of the subject relative to the medical instrument system, and/or periodically at spaced apart intervals as the disclosure is not limited in this fashion. Additionally, instances in which these processes are performed for multiple active implants disposed within the anatomy of a subject are also contemplated.

After registering the model to a reference frame of the medical instrument system, process 1500 may optionally proceed to stage 1540, in which real-time guidance to a target anatomy segment (e.g., target tissue like a cancerous lesion) may be provide. For example, the anatomical target may represent a cancerous lesion or otherwise diseased or problematic segment of tissue. The guidance may provide a graphical representation of the target tissue 970 to be treated or removed as well as a graphical representation of the real time position and orientation of the instrument 978 as it is being positioned during the procedure, see FIG. 7. The provided guidance may be implemented using pre-planned treatment guidance which may be determined in either in the optional planning stage 1450 in FIG. 4 and/or in a separate planning stage. Thus, the provided treatment guidance may include drug delivery guidance at 1542, tissue ablation guidance at 1544, tissue removal guidance at 1546, combinations of the foregoing, and/or any other appropriate type of treatment guidance as the disclosure is not so limited. Additionally, depending on the embodiment, the provided treatment guidance may either correspond to automated control of the medical instrument, semi-automated control of the medical instrument, and/or guidance provided to a user performing a manual procedure. In one such example, the model may be used with rendered virtual image(s) of a tool of the platform to provide real-time navigational guidance by displaying the relative positioning of the tool and the anatomical target to be treated within the registered anatomical model.

Depending on the type and number of procedures to be performed, the process 1500 may either end as indicated or it may repeat as needed.

It should be understood that any of the methods described above relative to FIGS. 11-13 and elsewhere in the current disclosure may be performed by at least one processor based on executable instructions encoded on at least one computer-readable storage medium. This may include implementations using a processor such as those described relative to a control system of the various embodiments a medical instrument system disclosed herein. Additionally, an operation including registration, confirmation of target tissue location, diagnosis or treatment of target tissue, and/or delivery of active implants may be performed using a system similar to that depicted in FIG. 1 and in FIG. 9 below.

FIG. 9 shows one example of a system that may be used with some embodiments herein, an exemplary teleoperated medical system for use in, for example, surgical, diagnostic, therapeutic, or biopsy procedures, is generally indicated by the reference numeral 1600. As shown in FIG. 9, the teleoperated system 1600 generally includes a teleoperational manipulator assembly 1602 for operating a medical instrument 1604 in performing various procedures on the subject. The assembly 1602 is mounted to or near an operating table O. A master assembly 1606 allows the clinician or surgeon S to view the interventional site and to control the slave manipulator assembly 1602. Master assembly 1606 generally includes one or more control devices for controlling the manipulator assemblies 1602. The control devices may include any number of a variety of input devices, such as joysticks, trackballs, data gloves, trigger-guns, hand-operated controllers, voice recognition devices, body motion or presence sensors, or the like.

The teleoperational assembly 1602 supports the medical instrument system 1604 and may include a kinematic structure of one or more non-servo controlled links (e.g., one or more links that may be manually positioned and locked in place, generally referred to as a set-up structure) and a teleoperational manipulator. The teleoperational assembly 1602 includes plurality of actuators or motors that drive inputs on the medical instrument system 1604 in response to commands from the control system (e.g., control system 1612).

The teleoperational medical system 1600 also includes a sensor system 1608 with one or more sub-systems for receiving information about the instruments of the teleoperational assembly. Such sub-systems may include a position sensor system (e.g., an electromagnetic (EM) sensor system); a shape sensor system for determining the position, orientation, speed, velocity, pose, and/or shape of the catheter tip and/or of one or more segments along a flexible body of instrument system 1604; and/or a visualization system for capturing images from the distal end of the catheter system. The sensor system may also include one or more sensors configured to detect a pose of an implant localization sensor of an active implant as detailed above.

The teleoperational medical system 1600 also includes a display system 1610 for displaying an image or representation of the surgical site and medical instrument system(s) 1604 generated by sub-systems of the sensor system 1608. Alternatively or additionally, the display 1610 may present images of the surgical site recorded pre-operatively or intra-operatively using image data from imaging technology such as, computed tomography (CT), magnetic resonance imaging (MRI), fluoroscopy, thermography, ultrasound, optical coherence tomography (OCT), thermal imaging, impedance imaging, laser imaging, or nanotube X-ray imaging. The pre-operative or intra-operative image data may be presented as two-dimensional, three-dimensional, or four-dimensional (including e.g., time based or velocity-based information) images or as images from models created from the pre-operative or intra-operative image data sets.

In some embodiments often for purposes of imaged guided surgical procedures, the display 1610 may display a virtual navigational image in which the actual location of the medical instrument 1604 is registered (i.e., dynamically referenced) with the model as detailed above to present the clinician or surgeon S with a virtual image of the internal surgical site from the viewpoint of the location of the tip of the instrument 1604. An image of the tip of the instrument 1604 or other graphical or alphanumeric indicators may be superimposed on the virtual image to assist the surgeon controlling the medical instrument. Alternatively, the instrument 1604 may not be visible in the virtual image.

In other embodiments, the display 1610 may display a virtual navigational image in which the actual location of the medical instrument is registered with preoperative or concurrent images to present the clinician or surgeon S with a virtual image of medical instrument within the surgical site from an external viewpoint. An image of a portion of the medical instrument or other graphical or alphanumeric indicators may be superimposed on the virtual image to assist the surgeon controlling the instrument 1604.

The teleoperational medical system 1600 also includes a control system 1612. The control system 1612 includes at least one memory and at least one computer processor (not shown), and typically a plurality of processors, for effecting control between the medical instrument system 1604, the operator input system 1606, the sensor system 1608, and the display system 1610.

During a virtual navigation procedure, the sensor system 1608 may be used to compute an approximate location of the instrument with respect to the subject anatomy. Various systems for using fiber optic sensors to register and display a medical implement together with preoperatively recorded surgical images, such as those from a virtual visualization system may be used. For example, U.S. patent application Ser. No. 13/107,562 (filed May 13, 2011) (disclosing “Medical System Providing Dynamic Registration of a Model of an Anatomical Structure for Image-Guided Surgery”), which is incorporated by reference herein in its entirety, discloses one such system.

Although the systems and methods of this disclosure have been described for use in the connected bronchial passageways of the lung, they are also suited for navigation and treatment of other tissues, via natural or surgically created connected passageways, in any of a variety of anatomical systems including the colon, the intestines, the kidneys, the brain, the heart, the circulatory system, or the like.

One or more elements in embodiments of the invention may be implemented in software to execute on a processor of a computer system such as control system 1612. When implemented in software, the elements of the embodiments of the invention are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit, a semiconductor device, a semiconductor memory device, a read only memory (ROM), a flash memory, an erasable programmable read only memory (EPROM), a floppy diskette, a CD-ROM, an optical disk, a hard disk, or other storage device. The code segments may be downloaded via computer networks such as the Internet, Intranet, etc.

Note that the processes and displays presented may not inherently be related to any particular computer or other apparatus. The required structure for a variety of these systems will appear as elements in the claims. In addition, the embodiments of the invention are not described with reference to any particular programming language. It will be appreciated that a variety of programming languages may be used to implement the teachings of the invention as described herein.

While the present teachings have been described in conjunction with various examples, it is not intended that the present teachings be limited to such examples. On the contrary, the present teachings encompass various alternatives, modifications, and equivalents, as will be appreciated by those of skill in the art. Accordingly, the foregoing description and drawings are by way of example only.

1. A system for performing a medical procedure, the system comprising:
an instrument for delivery of an active implant within a subject anatomy; and
at least one processor configured to:
receive an anatomical model of the subject anatomy, wherein the anatomical model is based on data captured prior to delivery of the active implant;
receive positional information for the active implant, wherein the positional information includes a pose of the active implant within the subject anatomy; and
establish the pose of the active implant as a reference within the anatomical model.

2. The system of claim 1, wherein the at least one processor is further configured to update the anatomical model based on the positional information for the active implant.

3. The system of claim 2, wherein the anatomical model is updated to reflect anatomical deformation of the subject anatomy.

4. The system of claim 1, wherein the instrument includes a delivery instrument localization sensor, and the positional information describing the pose of the active implant is received from the delivery instrument localization sensor.

5. The system of claim 4, wherein the delivery instrument localization sensor includes a shape sensing fiber or at least one EM sensor, and wherein the delivery instrument localization sensor is configured to provide a position and an orientation of at least a distal section of the instrument.

6. The system of claim 4, wherein establishing the pose of the active implant as the reference within the anatomical model includes registering the instrument to the anatomical model based on data from the delivery instrument localization sensor.

7. The system of claim 4, wherein the positional information from the delivery instrument localization sensor is a delivery location of the instrument when the instrument delivers the active implant to the delivery location.

8. The system of claim 1, wherein the instrument includes an imaging system, wherein the imaging system is configured to provide data used to update the anatomical model to reflect anatomical deformation of the subject anatomy.

9. The system of claim 1, wherein the positional information the describes a location of the active implant and is based at least in part on an input from a user.

10. The system of claim 1, further comprising the active implant, wherein the active implant includes an implant localization sensor.

11. The system of claim 10, wherein the at least one processor is further configured to register the subject anatomy to the anatomical model based on data from the implant localization sensor of the active implant.

12. The system of claim 10, wherein the anatomical model includes an anatomical target.

13. The system of claim 12, wherein the at least one processor is further configured to determine an implant location for delivery of the active implant, wherein the implant location is based at least in part on a location of the anatomical target.

14. The system of claim 12, wherein the at least one processor is further configured to determine a section of the subject anatomy for treatment based on the anatomical target.

15. The system of claim 14, wherein the at least one processor is further configured to provide guidance for treatment of the section of the subject anatomy.

16. The system of claim 15, wherein the guidance includes graphical indicators on or within the anatomical model, wherein the graphical indicators represent the section of the subject anatomy for treatment.

17. (canceled)

18. The system of claim 14, further comprising a therapeutic device configured for treatment of the section, wherein the therapeutic device includes a therapeutic localization sensor.

19. The system of claim 18, wherein the at least one processor is further configured to register the therapeutic device to the anatomical model.

20. (canceled)

21. The system of claim 18, wherein providing guidance for treatment of the section includes providing a representation of the therapeutic device within the anatomic model.

22. The system of claim 12, wherein the at least one processor is further configured to generate a planned path to the anatomical target in the anatomical model.

23-47. (canceled)
